(19) United States
(12) Patent Application Publication
(10) Pub. No.: US 2010/0256197 A1
(43) Pub. Date: Oct. 7, 2010

(54) Nicotine Dissolving Film With Or Without Menthol

(75) Inventors: Hanford N. Lockwood, JR., San Mateo, CA (US); Michele Lockwood, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Silver Eagle Labs NV, LLC, Carson City, NV (US)

(21) Appl. No.: 12/417,540

(22) Filed: Apr. 2, 2009

(51) Int. Cl. A61K 31/465

(52) U.S. Cl. 514/343

(57) ABSTRACT

A fast-dissolving film to be placed in the oral cavity of a person includes nicotine, or nicotine analog, with or without menthol, or menthol analog, that can be absorbed through the oral mucosa directly into the person's bloodstream for the purpose of reducing a temporary craving for nicotine during periods when smoking is not possible, or for therapeutic purposes, the film being thin and made from a low-moisture, non-tacky material that becomes dissolved when hydrated and that is sized so that the substrate can be placed in oral cavities of persons. From 0.1 mg to 10 mg nicotine, or a nicotine analog, and, optionally, menthol, or a menthol analog, are added to the substrate, which is then packaged in a flat, sealed container that can be unobtrusively placed into a pocket carried by the smoker, thereby making the container readily accessible to the smoker. During a period of nicotine craving encountered by the smoker when smoking is not possible, the film is removed from the container and placed into the smoker's oral cavity so that it contacts a mucosal membrane in the oral cavity of the smoker to thereby hydrate the substrate. The nicotine is thereby liberated from the film so that it can enter the smoker's bloodstream via the mucosal membrane to thereby temporarily satisfy the smoker's nicotine craving.

### BACKGROUND OF THE INVENTION

This invention relates to a new method for the oral mucosal delivery of nicotine for active smokers who are not allowed to smoke in restricted areas, such as at airports or in public transportation. There are now more than 2,200 municipalities in the United States with smoking restrictions. Of them, 461 totally ban smoking in private workplaces, government buildings, restaurants and/or bars. In Belmont, Calif., for example, residents who live in multi-unit buildings are not allowed to smoke in their homes. These restrictions impact the 60 million cigarette smokers in the U.S. who currently smoke and are not motivated to quit smoking.

Before a budding smoker realizes it, he becomes addicted to nicotine. This presents two problems. First, the nicotine habit is very difficult to overcome. The numerous treatments, prescriptions and other methods that are supposed to free the smoker from his nicotine addiction attest to this fact. Relatively few, however, are successful, so that many smokers continue the habit in spite of the fact that they have repeatedly attempted to end it.

A second problem encountered by active smokers is recurring nicotine cravings. Under normal circumstances, this is the moment when the smoker lights up another cigarette to satisfy his nicotine craving. At times, however, this is difficult or impossible to do. For example, smoking is strictly prohibited in public vehicles such as trains, airplanes and the like, as well as in many buildings, environments or simply while being amongst a group of non-smokers who resent smoking in their presence. There is little a smoker can presently do to overcome such short-term nicotine cravings other than move out of the space where smoking is not allowed, provided this is possible, or suffering through his nicotine craving until smoking becomes possible again.

Thus, the present invention provides an alternate means for taking the edge off of nicotine craving when a smoker cannot smoke. Conventional anti-smoking treatment frequently involves the use of nicotine gum, lozenges or dermal patches which are specifically geared toward ceasing smoking altogether and often contain doses of nicotine in quantities larger than those obtained from the typical cigarette. Such smoking cessation products require absolute abstinence from smoking cigarettes or using tobacco products of any kind. They are thus not suitable for persons who do not wish to stop smoking but who desire help to bridge them over a temporary nicotine craving time period.

There are further disposal and inconvenience issues with prior art nicotine cessation products that are absent from the present invention. Nicotine gum, patches and lozenges (if not consumed) contain enough residual nicotine to potentially poison children and pets and must be disposed of properly. The user of the nicotine gum has to follow a pattern of chew, park the gum along the gum line, chew, park, chew, park for a half hour or so until the nicotine is absorbed into the bloodstream. Chewing nicotine gum can also cause mouth, teeth or jaw problems because of the act of chewing. Sucking on a nicotine lozenge can be distracting to those around the user, while use of the nicotine gum or lozenge can be unacceptable in certain social situations. Because of the high amount of nicotine in the gum or lozenge, an overdose of nicotine can occur if more than one piece of gum or lozenge is chewed or consumed at the same time, or if many pieces are chewed or consumed one after another. Users of nicotine patches may experience irritation at the site of the patch on the skin due to adhesives in the patch or other skin sensitivities. The present invention overcomes the disadvantages encountered with the nicotine-containing products above. Instead, the present invention employs a thin, orally dissolvable nicotine-containing strip that is convenient to use, is socially acceptable and has no disposal issues to contend with.

To be effective, a nicotine-containing film or a strip constructed in accordance with the present invention, which may include menthol to facilitate the absorption of the nicotine via the mucosal membrane into the patient's bloodstream, must be readily available and easy to carry so that it can be used at any time and/or any place, and it must be sufficiently discrete and non-bulky so that the strips can be carried by the smoker in pockets of his clothing, briefcases, purses and the like which are within easy reach of the smoker should the need for a nicotine bridge arise. Thus, in accordance with the present invention the nicotine is contained in flat, thin films or strips that can be orally taken while they remain effectively hidden from open view.

Nicotine is an alkaloid found in the nightshade family of plants (Solanaceae) such as tobacco. In low concentrations (an average cigarette contains 10 mg of nicotine, only 1 to 2 mg of which are absorbed into the bloodstream through the lungs), nicotine acts as a stimulant and is one of the main reasons smokers become addicted to cigarettes. Studies by the National Institute on Drug Abuse [NIDA] have shown that nicotine activates reward pathways—the circuitry within the brain that regulates feelings of pleasure and euphoria.

This invention is intended to ease the discomforts associated with nicotine craving when a smoker is temporarily prevented from smoking. It consists of a pleasant tasting, rapidly dissolving film which contains a relatively low dosage of nicotine. The film is placed on the back of the tongue or against the roof of the mouth. Within seconds the film dissolves, and the nicotine is absorbed into the bloodstream. Within a few seconds of entering the bloodstream, nicotine reaches the brain. For smokers who begin to crave nicotine and cannot light up, this invention provides immediate relief. The smoker can also regulate the quantity of nicotine by consuming a successive number of nicotine dissolving films according to his own needs.

Another advantage of the nicotine dissolving film is that it lacks the carcinogenic substances found in cigarettes and the damaging effects of particulates inhaled into the lungs.

Research studies have also observed that nicotine may be of therapeutic value to patients who suffer from a wide range of diseases and mental illnesses, such as Parkinson's disease, Alzheimer's disease, schizophrenia, and attention-deficit/hyperactivity disorder, as well as a means of appetite-control to promote weight loss.

Research has also shown that the addition of menthol to a drug delivery system expedites the absorption of the drug into the bloodstream. Menthol is an organic compound made synthetically or obtained from peppermint or other mint oils. It is a pungent, waxy, crystalline substance, clear or white in color. Menthol has local anesthetic and counterirritant qualities, as well as local vasodilation characteristics. Because of its ability to rapidly penetrate the mucosal membranes in the nose and throat, menthol is widely used as a decongestant, as well as a remedy for minor sore throats. The local vasodilation characteristic of menthol has been shown to be effective in the rapid delivery of ibuprofen, nitroglycerin, and other pharmacologically active ingredients. Clinical studies have shown that menthol can dramatically increase the transdermal delivery of medications and/or herbal supplements. In the nicotine-supplying strips or films of the present invention, menthol acts as a vasodilator allowing the rapid absorption of nicotine into the bloodstream through the oral mucosa, thereby enabling the smoker to obtain the effects of a single cigarette at a lower nicotine content and at a faster rate.

The methods for delivering medicines or drugs into the human system through the skin or mouth in the prior art reveal fast dissolving oral films for breath fresheners, chewing gum or lozenges, drinking beverages, or applying gel or ointment compositions to the skin.

Thus, U.S. Pat. Nos. 6,923,981 and 6,596,298 by Leung et al. disclose fast-dissolving orally consumable films used to deliver breath deodorizing agents, antimicrobial agents and salivary stimulants to the oral cavity. The films can also be used to deliver pharmaceutically active agents. Leung discloses an improvement to films in the prior art, as well as the processes for making them.

U.S. Pat. No. 6,893,654 by Pinney discloses a two-stage transmucosal medicine delivery system provided in chewing gum or lozenge form to deliver a craving reduction substance through the mucosal tissues in the mouth. The system and apparatus can be adapted to reduce cravings for nicotine, alcohol, food and drugs (such as cocaine, opiates and the like). Likewise, U.S. Pat. No. 6,344,222 by Cherukuri et al. describes a chewing gum delivery system with nicotine, a gum base and a buffer system with an improved release rate for nicotine.

U.S. Pat. No. 6,559,180 by Busiashvili reveals the effect of menthol on the dosage of nitroglycerin. When used in combination with menthol, the dosage of nitroglycerin can be lowered without sacrificing effectiveness, but reduces the common side effects of taking nitroglycerin such as headache and fainting.

U.S. Pat. No. 6,479,076 describes a nicotine delivery system through the skin of the user whereby a water-insoluble vinylpyrrolidone copolymer in the form of a gel, ointment, solution, suspension or film is applied to the skin.

U.S. Pat. No. 6,268,386 by Thompson teaches the delivery of nicotine through the oral consumption of fluids.

### SUMMARY OF THE INVENTION

The nicotine delivery systems on the market today are designed to aid a smoker in the cessation of smoking. The nicotine patch, the nicotine gum and the nicotine lozenge are all designed to be part of a stop-smoking program. The present invention is directed toward the smoker who enjoys the ritual of smoking and does not want to quit. While not an obvious ritual in itself, the present invention gives a committed smoker relief from nicotine craving between smoking opportunities. This characteristic distinguishes the nicotine dissolving film or strip from all other nicotine delivery systems on the market today.

This invention is an improvement over the prior art in that it delivers nicotine or a nicotine analog (in the context of this invention, a nicotine analog is a compound with a structure similar to that of pure nicotine but differing from pure nicotine with respect to certain components, but which act metabolically similarly) in a rapidly dissolving film with or without menthol for the purpose of temporarily reducing the craving for nicotine when a smoker cannot smoke without the carcinogenic risk attendant with the chewing of tobacco or use of snus, a moist powder tobacco product that is consumed by placing it under the upper lip for extended periods of time. The advantages of the present invention over the prior art include convenience, discretion, lack of concern with respect to disposal issues, and a healthier alternative to cigarette smoking while mitigating the craving for nicotine.

It is therefore an object of the present invention to provide a socially acceptable mechanism that delivers a small amount of nicotine through the oral mucosa in the user's mouth. An important feature of the present invention is that the nicotine dissolving film delivery system can be discreetly used by a smoker anytime anywhere. The amount of nicotine delivered is precise and easily controlled by the smoker.

The present invention utilizes the unique features of consumable films that are low in moisture, non-tacky in texture and substance, and rapidly dissolvable by composition. Pure nicotine or a nicotine analog from 0.1 mg to 10 mg with or without from 1% to 10% by weight menthol or menthol analog (a chemical compound similar to menthol which has a similar action metabolically) is mixed or injected into the film before the drying process. Rolls of nicotine-injected film can then be stored in protective wrappers until cut by machine and packaged in individual foil wrappers or bundled in cassettes for commercial use. Both the rolls of nicotine-injected film and the individually foil-wrapped nicotine strip can be stored at room temperature.

Another advantage is that the present invention requires no special handling or storage. The preferred individually foil-wrapped packaging of the present invention provides stability and freshness to the nicotine delivery system. A side benefit of the pleasant-tasting film is the improvement in malodorous breath.

The present invention not only provides an effective and convenient way for a smoker to satisfy a temporary nicotine craving during periods when he cannot smoke, it also provides a convenient and inconspicuous way of facilitating the application of a nicotine boost by forming the nicotine-bearing strips so that they can be readily placed in the user's oral cavity while keeping the film ready for use at any time and anywhere. The nicotine-bearing film of the present invention is packaged in a sealed container (to prevent the escape of nicotine and/or menthol) which is flat so that it can be readily and inconspicuously placed in pockets, purses, briefcases, wallets and the like on or next to him. Preferably, the strips are individually wrapped in peel-off packages which are widely available in the marketplace. In addition, and/or alternatively, the packages can be placed in dispensers holding multiple nicotine film-carrying packages and/or they can be placed directly into appropriately constructed dispensers.

The person will normally carry at least one, and typically a small supply, of nicotine-bearing films on him, for example in his pockets. When a nicotine craving occurs, regardless of the place or time, the smoker simply reaches into his pocket, for example, discretely removes the package therefrom, peels back the sealing cover, and then places the film in his oral cavity for dissolution of the film and satisfying the nicotine craving.

### BRIEF DESCRIPTION OF THE DRAWINGS

FIG. 1 is a perspective, front elevational view of a flat container for sealingly storing the nicotine-containing film which has a peel-back sheet to provide access to the film; and

FIG. 2 is a side elevational view, in section, of a container for holding several nicotine-bearing films constructed in accordance with the present invention.

### DESCRIPTION OF THE PREFERRED EMBODIMENTS

Referring to the drawings, the present invention provides smokers with relief from the craving for nicotine when they are unable to smoke cigarettes. Smokers on public transportation, in public buildings, in restaurants or in bars, to name a few, find it increasingly difficult to satisfy their craving for nicotine when smoking is not possible. The present invention employs a nicotine-carrying film or strip that utilizes dissolving film technology as the delivery system of pure nicotine or a nicotine analog, with or without menthol or menthol analog, to relieve the user's nicotine craving when he is prevented from having a cigarette, a pipe or chewing tobacco, for example. Although the present invention is not designed to and does not stop a person from smoking, the nicotine-carrying film of the invention offers relief from nicotine craving between smoking opportunities.

The nicotine dissolving film or strip is a convenient way of delivering nicotine to the smoker that has advantages over the current nicotine delivery systems, such as nicotine patches, nicotine gum or nicotine lozenges, which are designed and used as stop-smoking aids and not to just temporarily satisfy a temporary nicotine craving. The amount of nicotine delivered by the film to the smoker is typically less than the amount delivered from inhaling a cigarette. A typical cigarette contains approximately 10 mg of nicotine, only 1 to 2 mg of which are absorbed into the bloodstream during inhalation. The dissolving nicotine-bearing film of the present invention rapidly disintegrates on the user's tongue or the roof of the mouth, allowing nicotine to be quickly absorbed via the mucosal membranes into the bloodstream, thereby offering immediate relief to the nicotine-craving smoker. The dissolving film leaves no residue which the smoker must discard, which is a distinct advantage over nicotine gums, patches or lozenges.

In its presently preferred embodiment of the invention, a combination of pure nicotine, or a nicotine analog, with a readily dissolving film, with or without menthol or menthol analog, delivers the nicotine to the user's bloodstream. The dissolving film is low in moisture, non-tacky in texture, and designed to dissolve rapidly when in contact with the moisture in a smoker's mouth. The dissolving film is cut during the manufacturing process as a strip between 1.5 to 3 mils thick and square or rectangular in shape with a surface area from about 0.5 to about 2 square inches. The size of the preferred embodiment is a strip 0.8 inches wide by 1.25 inches long with a weight of 55 mg. The nicotine content of the strip is from about 0.1 to about 10 mg depending on the intended use of the product. When using the nicotine-bearing film of the present invention as a bridge between smoking opportunities, the film will typically have from about 0.5 mg to about 1.2 mg of pure nicotine, or a nicotine analog, and between 5% to 7% by weight menthol, or menthol analog. In some cases higher levels of nicotine content in the dissolving film delivery system can be used for therapeutic purposes, such as relief for patients with post-traumatic stress disorder, bipolar disorder, major depression and other mental illness, including schizophrenia.

The dissolving film is made by a process in which the ingredients are thoroughly mixed in an aqueous solution that contains both the constituents of the dissolving film and the active ingredients, namely nicotine, or a nicotine analog, and, if desired, menthol, or a menthol analog. The aqueous solution is then dried at the appropriate temperature and over the needed time interval to attain the desired film thickness and ingredient composition. The dried film is then cut to the dimensions of the desired strip size containing the appropriate dosage of the active ingredients.

In the preferred embodiment, the dissolving film can contain hydroxypropyl methylcellulose, microcrystalline cellulose, carrageenan, alpha bisabolol, polysorbate 80, glycerin, artificial sweeteners, flavoring, food coloring, combined with the active ingredient of pure nicotine or a nicotine analog with or without menthol or menthol analog. The hydroxypropyl methylcellulose, microcrystalline cellulose, carrageenan, alpha bisabolol, glycerin, and polysorbate 80 make up the base substrate of the dissolving film. This substrate is vegetarian based allowing the widest possible use by the smoking population. In addition the use of artificial sweeteners, such as sucralose, allows diabetic smokers to use this invention safely. The flavoring of the nicotine dissolving film is designed to disguise or mask the taste of pure nicotine or a nicotine analog. In the preferred embodiment, a peppermint flavoring is used; however, any other strong mint or natural flavoring can be used to cover up the taste of nicotine. Although in the preferred embodiment of this invention the constituents of the film are vegetarian based, it is also possible to use an animal product based substrate to which an artificial sweetener, appropriate flavoring, food coloring, etc. as well as pure nicotine, or a nicotine analog, with or without menthol, or menthol analog, are added during the manufacture of the film.

The finished nicotine-carrying film of the present invention is hygroscopic and can experience off-gassing of ingredients which can degrade the product over time if it is not packaged properly. One packaging option is the use of plastic cassettes which can hold, for example, up to 24 nicotine-carrying films. The cassette should be only slightly larger than the films to minimize the air volume in the cassette. The cassette is then packaged in a blister pack with plastic on one side and a foil seal between the cassette and the cardboard backing of the blister pack. This packaging is air-tight until the cassette is removed from the blister pack. Once the cassette is removed from the blister pack and the first nicotine-carrying film is removed, however, the remaining films should be consumed in a relatively short period of time before the strips absorb too much moisture and degrade. Such packaging carries the risk, however, of simultaneously removing two or more strips as they begin to stick together due to moisture absorption, causing the user to receive a greater dose of nicotine than desired or expected.

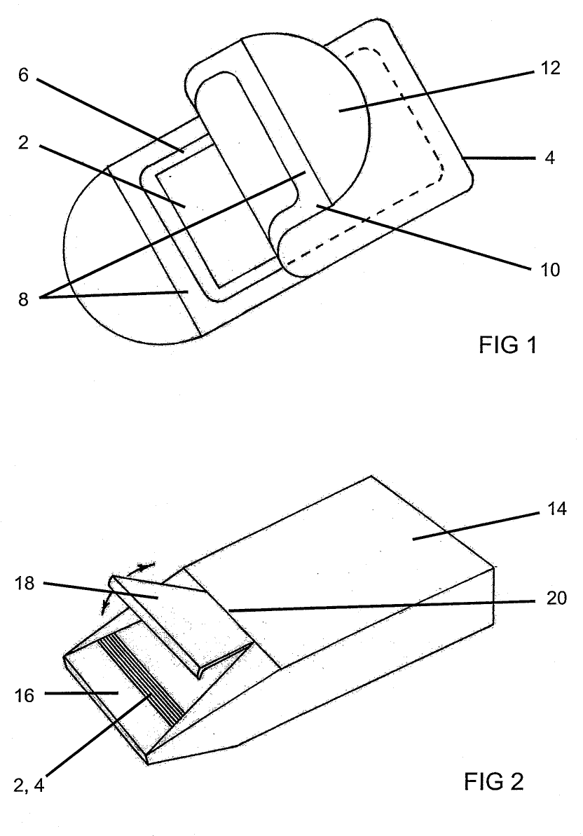

Referring to FIG. 1, in a presently preferred embodiment a nicotine-carrying film 2 constructed in accordance with the present invention as above described has a rectangular outline and is thin (preferably in the range between about 1.5 to 3 mils) so that it is flexible and can be readily placed in a person's oral cavity. The film is placed inside a container constructed of a gas-impervious material, such as, for example, any one of a large variety of plastics or foil used for packaging, and has a depth (not separately shown in FIG. 1) sufficient to readily accommodate one film. A top 6 of the container is open, has outwardly extending flanges 8 that surround the film inside the container, and includes a peel-back sheet or membrane 10 that is sealingly secured, e.g. bonded, to the lateral flanges 8 of the container. The bond of the flexible membrane keeps the interior of the container sealed from the exterior, thereby preventing a degassing of components of the film and/or its contamination while the bond is sufficiently weak so that the membrane is readily pulled back to expose the film for removal from the container. To facilitate the peel-back of the membrane, it may include a tab 12 where the membrane can be grasped for pulling it off the flanges of the container. The thickness of the container is kept thin to facilitate carrying it in pockets and the like. Its thickness can be as small as 1 to 3 mm to accommodate the thickness of the film plus the thickness of the container walls on top of and below the strip in the container.

In use, the smoker places one or more of the sealed containers 4 into his pocket, for example, and carries the strips with him at all times so that the strip is available when needed. When the user experiences a nicotine craving and is not in a position to smoke (or chew tobacco), for example, he places his hand into the pocket or briefcase, peels back membrane 10 from container 4, and then slides the film out of the opened container with his fingers. By slightly moistening his fingers prior to touching the film, the film will adhere to the user's finger, which facilitates placing the film in his oral cavity.

To provide an extra supply of nicotine-carrying films, for example when the user anticipates a prolonged period of being unable to smoke in order to satisfy a nicotine craving, such as on intercontinental flights, a larger number of nicotine-carrying films can be placed into a multi-film holding cartridge 14 as illustrated in FIG. 2. One end 16 of the cartridge is open, and a cover 18 is provided for closing the open cartridge end. A pivot 20 can be provided to secure the cover to the cartridge to facilitate closing of the cartridge after use.

As previously mentioned, a number of nicotine-bearing films can be placed directly into the cartridge. In such an event, the cartridge or cassette will be sealed, for example by enclosing it inside an air-impervious pouch, a blister package or the like. The disadvantage of this arrangement is that it will typically be necessary to use up the entire supply of films inside the cartridge over a limited period of time to prevent their degradation over time.

Alternatively, the films placed inside the cartridge can themselves be packaged into sealed container 4 illustrated in FIG. 1, and a plurality of such containers, each holding one nicotine-carrying film, is placed inside the cartridge. When the user requires a strip, he opens cover 18, slides out one of the packages, and then opens the sealing membrane 10 on the container as above described.

The thickness of cartridge 14 shown in FIG. 2 is preferably kept as thin as possible for holding the desired number of films so that the cartridge too can be carried by the user at all times to assure its availability when and where needed.

What is claimed is:

1. A thin film for the delivery of a desired substance to a person's bloodstream via a mucosal membrane comprising a thin substrate of a size to be received in the person's oral cavity and made of a low-moisture, non-tacky material which rapidly dissolves when in contact with the mucosal membrane, and from 0.1 mg to 10 mg of nicotine or nicotine analog so that, upon dissolution of the substrate, the nicotine enters the bloodstream through the mucosal membrane of the person.

2. A thin film according to claim 1 wherein the nicotine present in the substrate is one of pure nicotine or a nicotine analog.

3. A thin film according to claim 1 including between 1% and 10% by weight of menthol or menthol analog in the substrate.

4. A thin film according to claim 1 wherein the substrate is made of vegetable ingredients only.

5. A thin film according to claim 1 wherein the substrate includes some animal by-products.

6. A thin film according to claim 1 wherein the substrate includes an artificial sweetener.

7. A thin film according to claim 1 wherein the substrate includes a flavoring substance.

8. A thin film according to claim 1 including a foil wrap exterior cover sealing the film from the environment.

9. A thin film according to claim 1 including a container having a thickness only slightly greater than a thickness of the film, and a removable cover placed over the container and sealing the film in the container from the exterior thereof.

10. A method of providing a temporary nicotine bridge for a smoker during periods when the smoker cannot smoke comprising
providing a thin substrate made from a low-moisture content, non-tacky material that becomes dissolved when hydrated and that is sized so that the substrate can be placed in oral cavities of persons,
adding from 0.1 mg to 10 mg nicotine, or a nicotine analog, to the substrate,
during a period of nicotine craving encountered by the smoker, placing the substrate including the nicotine or nicotine analog against a mucosal membrane of the smoker in his oral cavity, and
letting the substrate dissolve while it is in contact with the mucosal membrane to thereby liberate the nicotine or nicotine analog from the substrate so that the nicotine or nicotine analog can enter the smoker's bloodstream via the mucosal membrane.

11. A method of enabling smoker to temporarily bridge a nicotine craving between a time when and at a place where smoking is not possible and when the smoker can smoke again, the method comprising
providing a thin substrate made from a low-moisture, non-tacky material that becomes dissolved when hydrated and that is sized so that the substrate can be placed in oral cavities of persons,
adding from 0.1 mg to 10 mg nicotine, or a nicotine analog, to the substrate, packaging the substrate in a flat container,
applying a sealing, readily removable membrane over the container to seal the substrate from the environment,
unobtrusively placing the sealed container into a pocket carried by the smoker so that the container is readily accessible to the smoker,
during a period of nicotine craving encountered by the smoker when smoking is not possible, removing the substrate from the container,
placing the substrate into the smoker's oral cavity and contacting the substrate with a mucosal membrane in the oral cavity of the smoker to thereby hydrate the substrate, and
waiting for nicotine liberated from the dissolving substrate to enter the smoker's bloodstream via the mucosal membrane to thereby temporarily satisfy the smoker's nicotine craving.

12. A method according to claim 11 wherein removing the substrate comprises pulling the membrane at least partially off the container and thereafter removing the substrate from the container.
